(19) United States
(12) Patent Application Publication
(10) Pub. No.: US 2003/0199917 A1
(43) Pub. Date: Oct. 23, 2003

(54) Thrombus treatment with emboli management

(76) Inventors: Mark B. Knudson, Shoreview, MN (US); Timothy R. Conrad, Eden Prairie, MN (US)

Correspondence Address: MERCHANT & GOULD PC, P.O. BOX 2903, MINNEAPOLIS, MN, 55402-0903, US

(21) Appl. No.: 10/128,120

(22) Filed: Apr. 22, 2002

(51) Int. Cl. A61M 29/00

(52) U.S. Cl. 606/200

(57) ABSTRACT

A method for treating a body lumen having a natural occlusion at least partially occluding a flow of body fluid in the lumen includes obstructing the lumen with an artificial occlusion distal to the natural occlusion. The natural occlusion is ablated in a process which may create a plurality of emboli of the natural occlusion on a proximal side of the artificial occlusion. The emboli are removed from the lumen and, subsequently, the artificial occlusion is removed.

### I. BACKGROUND OF INVENTION

1. Field of the Invention

This invention pertains to a method and apparatus for protecting body tissue during the removal of obstructions from a body lumen. More particularly, this invention pertains to methods and apparatus for reducing the possibility of emboli migrating distal to an obstruction when removing the obstruction from a body lumen.

2. Description of the Prior Art

From time to time, a body lumen may develop a natural occlusion restricting fluid flow through the lumen. For example, blood vessels such as arteries may develop blockages for a variety of reasons. Plaque formation on an interior wall of the artery may result in thrombus formation. Such thrombus may fully or partially occlude the artery.

When an artery is occluded, blood cannot flow freely distal to the occlusion. This results in a lack of oxygenated blood flowing to tissue being served by the artery. In the case of a coronary artery, such blockage can lead to ischemia or infarction of the heart muscle. In the event of a cerebral muscle, such blockage can result in a cerebral ischemia or stroke. In peripheral vessels, the health of limbs is put at risk.

Numerous therapies are used to treat occluded vessels. For example, drug therapies use clot-ablating chemical agents to break up a clot. See, e.g., U.S. Pat. No. 5,925,016. Such therapies may be used in combination with shock waves (U.S. Pat. No. 5,709,676).

Balloon angioplasty involves placement of a balloon on a tip of a catheter within the clot and expanding the balloon to urge the clot against the walls of the blood vessel in order to open the blood vessel. Stenting used in conjunction with or independent of balloon angioplasty involves placing a stent in the occluded area and expanding the stent to open the occlusion and urge the stent against the wall of the lumen.

Mechanical ablation includes a number of different techniques for placing a mechanical agitator in the region of the clot to break the clot open. The mechanical agitation could be a rotary bit acting against the clot to break it up. Examples of such are shown in U.S. Pat. Nos. 5,376,100; 4,857,045 and 4,646,736. Also, an ejected fluid (such as water) can be used as a jet to breakup the clot. See, e.g., U.S. Pat. No. 5,370,609. Aspiration and mechanical thrombectomy are reviewed in Morgan et al., “Percutaneous thrombectomy: a review”, European Radiology, pp. 205-217 (January 2002). Various mechanical thrombectomy devices are reviewed and compared in Kasirajan et al., “The use of mechanical thrombectomy devices in the management of acute peripheral arterial occlusive disease”, J. Vascular and Interventional Radiology, pp. 405-411 (April 2001).

Also, application of energy has been attempted to break-up a clot. For example, U.S. Pat. No. 5,058,570 teaches use of ultrasound for such purpose.

Whenever a blood vessel is treated to remove an obstruction, a risk exists that minute pieces of the obstruction (referred to as emboli) may break off and flow distal to the obstruction. See, e.g., Titus, et. al., “Distal embolization during mechanical thrombolysis: rotational thrombectomy vs. balloon angioplasty”, Catheterization and Cardiovascular Diagnosis, pp. 279-285 (April 1990). Such emboli may in turn obstruct the blood vessel or any of its branching vessels distal to the original obstruction. Such events continue or compound the original problem of ischemia.

Numerous techniques have been attempted to manage the consequence of emboli formation. For example, mechanical filters have been developed to be placed distally of an obstruction in order to trap emboli during treatments of lumen obstructions. Examples of such mechanical structures are shown in U.S. Pat. Nos. 5,941,896; 5,911,734; 5,695,519 and 6,066,149.

Other techniques for capturing emboli include aspiration to draw emboli proximally away from an occlusion and into a catheter. See, e.g., U.S. Pat. Nos. 5,370,609; 4,857,045 and 5,938,645. When clots are being removed, balloons may be inflated distal to the clot to control emboli flow. See, e.g., U.S. Pat. Nos. 6,022,336; 5,925,016 and 5,059,178.

Not withstanding prior art attempts to manage uncontrolled emboli formation, a continuing need exists in the art for preventing the distal travel of emboli. For example, some of the prior art apparatus cannot capture all of the emboli and are typically relatively stiff devices, which cannot be easily manipulated into position for treatment of an occlusion.

Filters and other devices are frequently limited to larger vessels. In addition, the opening of the filter can cause significant damage if mistakenly opened in too small of a vessel. Also, the act of passing a large catheter with such devices can, in itself, cause emboli.

The incomplete visualization of a thrombectomy procedure can present serious risks with prior art devices. For example, a guide wire may be passed through a fully occluded site in a lumen and a balloon or a filter may be opened near the tip of the guide wire distal to the occlusion. Placement of a guide wire is commonly performed under fluoroscopy where a radiopaque dye is injected into the blood stream. In the case of a complete occlusion, the dye cannot flow distal to the occlusion and the physician is not capable of visualizing the tip of the catheter distal to the obstruction. The catheter tip may have migrated into a small branching vessel (such as a septal diffusing vessel branching from a coronary artery). If a filter or a balloon were to be inflated in such a small vessel, the vessel may rupture.

In many patients, vessels may be extremely fragile and small. This is particularly true in the case of cerebral vessels. Also, certain patient diseases (e.g., diabetes) may make vessels particularly small or fragile. The size and fragile nature of these vessels may preclude the use of certain techniques (such as the placement of filters or balloons) in order to avoid vessel rupture. If they were to rupture, a thrombotic event (stroke, acute myocardial infarction) would be converted into a catastrophic hemoragic event.

It is an object of the present invention to provide a method and apparatus for controlling emboli flow distal to an original obstruction site.

### II. SUMMARY OF THE INVENTION

According to a preferred embodiment of the present invention, a method is disclosed for treating a body lumen having a natural occlusion at least partially occluding a flow of body fluid in the lumen. The method includes obstructing the lumen with an artificial occlusion distal to the natural occlusion. The natural occlusion is ablated in a process which may create a plurality of emboli of the natural occlusion on a proximal side of the artificial occlusion. The emboli are removed from the lumen and, subsequently, the artificial occlusion is removed. In a further embodiment of the present invention, an apparatus is disclosed for treating a body lumen having a natural occlusion. The apparatus includes a delivery member sized to be passed through the body lumen proximal to the natural occlusion and having a distal end adapted to be passed through the natural occlusion. The delivery member includes a delivery port adjacent the distal end for delivery of an artificial occlusion into the lumen distal to the natural occlusion. A still further embodiment of the present invention includes a kit for treating a body lumen having a natural occlusion. The kit includes an occlusion-creating member for occluding the lumen with an artificial occlusion distal to the natural occlusion. An ablator ablates the natural occlusion to create a plurality of emboli of the natural occlusion on a proximal side of the artificial occlusion. An emboli-removing member removes the emboli from the lumen.

### III. BRIEF DESCRIPTION OF THE DRAWINGS

FIG. 1 is a side sectional, schematic view of a blood vessel containing a natural occlusion;

FIG. 2 is the view of FIG. 1 with a guide wire passed through the occlusion and with portions of the blood vessel, guide wire and occlusion shown in phantom lines to illustrate obstructed vision of a physician attempting to visualize a procedure under fluoroscopy;

FIG. 3 is the view of FIG. 2 (shown in solid lines) following formation of an artificial occlusion distal to the natural occlusion;

FIG. 4 is the view of FIG. 3 showing mechanical ablation of the natural occlusion and resulting formation of emboli;

FIG. 5 is the view of FIG. 4 following complete ablation of the natural occlusion and showing removal of the emboli;

FIG. 6 is the view of FIG. 5 following complete removal of the emboli and showing an optional embodiment for dissolving the artificial occlusion;

FIG. 7 is the view of FIG. 6 following complete dissolving of the artificial occlusion;

FIG. 8 is the view of FIG. 3 (without showing a guide wire) and showing an alternative placement of an artificial occlusion directly abutting a distal side of a natural occlusion;

FIG. 9 is a side sectional view of a guide wire for delivery of an artificial occlusion distal to a natural occlusion;

FIG. 9A is a view taken along line 9A-9A in FIG. 9; and

FIG. 10 is a schematic, side sectional view of a coronary artery and parallel-aligned coronary vein with the coronary artery containing a natural occlusion; and

FIG. 11 is the view of FIG. 10 with an artificial occlusion applied to the coronary artery distal to the natural occlusion according to an alternative embodiment of the invention.

### IV. DESCRIPTION OF THE PREFERRED EMBODIMENT

Referring now to the several drawing figures in which identical elements are numbered identically throughout, a description of a preferred embodiment of the present invention will now be provided. As will be apparent to one of ordinary skill in the art, the present invention can be applicable to treatment of any occlusion in any body lumen. For ease of description, the present invention will be described in a preferred embodiment for treatment an occlusion in a blood vessel such as a cerebral artery or coronary artery.

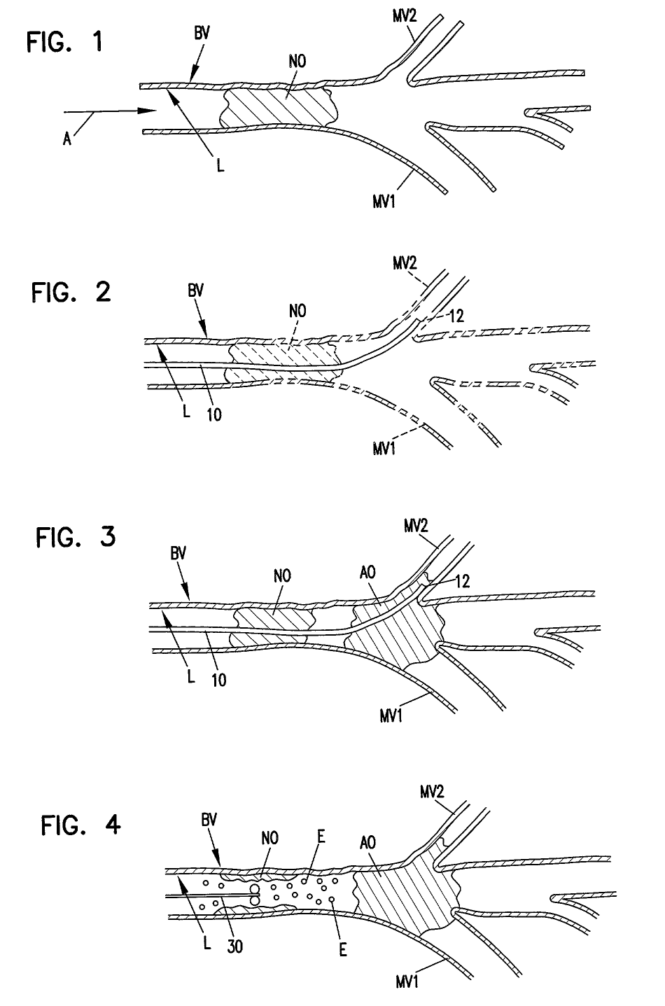

With initial reference to FIG. 1, a blood vessel BV is shown. The blood vessel BV could be a cerebral artery, coronary artery or any other blood vessel, which contains a natural occlusion NO blocking blood from flowing through a lumen L in the normal direction indicated by arrow A in FIG. 1.

As illustrated in FIG. 1, the blood vessel BV includes branching vessels such as a first minor vessel MV1 and a second minor vessel MV2. In the example of FIG. 1, branching vessels MV1 and MV2 are located distally (i.e., downstream) of the natural occlusion NO.

The natural occlusion NO can be any naturally occurring occlusion in the blood vessel BV. For example, plaque may form on the wall of the blood vessel BV causing occlusion itself or such plaque may rupture resulting in a soft thrombus or clot fully occluding the vessel BV. In FIG. 1, the natural occlusion NO is shown as a complete occlusion of the lumen L. It would be appreciated that the present invention may also be used where the natural occlusion NO only partially occludes the blood vessel lumen L.

FIG. 2 illustrates the placement of a guide wire 10 through the lumen L with a distal end 12 of a guide wire 10 projecting through the soft thrombus of the natural occlusion NO. FIG. 2 illustrates an occurrence where the distal end 12 has migrated into the smaller second minor vessel MV2 (e.g., a septal perfusing vessel of a coronary artery).

Flexible guide wires are well known and an example of such is shown in U.S. Pat. No. 5,437,288. U.S. Pat. No. 6,193,676 teaches a guide wire for use in total occlusions.

Guide wires have soft flexible distal tips to reduce the probability of trauma to a blood vessel as the guide wire tip is advanced by a physician through the patient's blood vessel to a desired site. Guide wire 10 of the present invention differs from the guide wires of the prior art as will be later described.

In the case of a thrombus acting as a natural occlusion NO, skilled physicians can advance the soft tip guide wire through the thrombus as illustrated in FIG. 2. Such a procedure is performed under fluoroscopy where a contrast media (such as a radiopaque dye) is injected into the blood stream. In the event of a complete occlusion such as that illustrated in the figures, the blood cannot carry the dye distal to the occlusion. Therefore, the portions of the blood vessel distal to the proximal side of the occlusion NO are not susceptible to visualization by the physician.

Commonly, the guide wires are radiopaque and are susceptible to visualization even though they may reside in a portion of the blood vessel not susceptible to visualization. This is illustrated in FIG. 2 where the portion of the blood vessel proximal to the natural obstruction NO is shown in solid lines. The natural obstruction NO and portions of the blood vessel BV distal to the natural obstruction NO are shown phantom lines. The guide wire 10 is shown in solid lines throughout.

As illustrated in FIG. 2, the distal end 12 of the guide wire 10 has migrated into the smaller second minor vessel MV2. Since the minor vessel MV2 itself is not subject to visualization, the physician may inaccurately conclude that the distal end 12 resides in the main lumen L of the vessel distal to the natural occlusion NO. In certain prior art procedures, a guide wire may be provided with a balloon at its distal tip. Alternatively, a balloon-tipped catheter (with or without a stent or an expanding mechanical filter) may be passed over the guide wire and the balloon may be expanded. If this were to occur in the situation depicted in FIG. 2, the balloon, stent or mechanical filter would be expanded within the very narrow minor vessel MV2 creating the risk of rupture of the minor vessel MV2. Such rupture could be catastrophic. From the remainder of the present description it will be appreciated that it is immaterial to the present invention if the physician is aware that the distal tip 12 has migrated into a narrow branching vessel MV2.

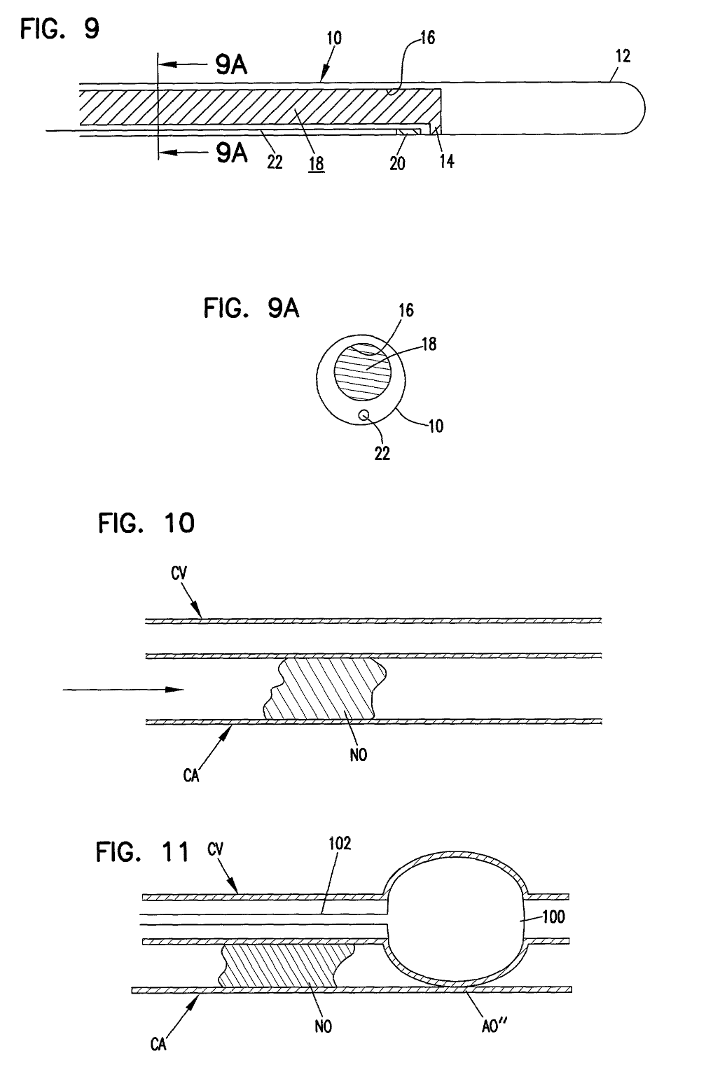

With reference to FIGS. 3 and 9, the guide wire 10 of the present invention is modified from those of the prior art to have an internal cavity 16 in communication with a side delivery port 14 adjacent a highly flexible distal tip 12. The cavity 16 contains a volume of material 18 which can be ejected through the port 14 at the election of the physician. If desired, the port 14 can be sealed with a seal (not shown) which is selected to rupture when the material 18 is being ejected.

The cavity 16 may be an extension of a lumen along the entire length of the guide wire 10. A supply of the material 18 may be injected into a proximal end (not shown) of the guide wire 10 and travel along the length of the guide wire 10 for discharge through the port 14. Alternatively, a metered amount or bolus of the material 18 may be residing in the cavity 16 adjacent the port 14 and with a back pressure of fluid (such as saline water) proximal to the material 18 to operate under pressure to eject the material 18 through the port 14.

Not shown in FIG. 9, the guide wire 10 can have a second lumen with a second discharge port near the distal tip 12 for ejecting a contrast media into the lumen of the blood vessel BV distal to the natural occlusion NO.

The material 18 contained within the guidewire 10 is a material for forming an artificial occlusion within the blood vessel BV. Preferably, the material 18 is a material selected to swell following discharge from the port 14 and expand within the blood vessel. It is desired that the material 18 can seal against the blood vessel walls with a pressure sufficient to block blood flow past the swelled material AO. The material is conformal in that it flows into conforming opposition to the walls of the vessel BV.

Such a material 18 could be a hydrogel contained in an unswelled state within the guide wire 10 and which swells in the presence of water within the blood vessel upon ejection from the port 14. Other materials could be so-called “smart polymers” or “smart hydrogels” which can swell in response to a number of different parameters including the presence of water, selected pH or application of an electrical current to more selectively control the timing of the swelling. The electrical current could be provided by leads (not shown) on the surface of guide wire 10.

In the embodiment shown, the material 18 is a hydrogel carried in the guide wire 10 in an unswelled state and which swells in response to the presence of water in the blood vessel BV. After ejection of the material 18 from the port 14, the hydrogel swells to form an artificial occlusion AO as illustrated in FIG. 3.

It will be noted that the fluid of the hydrogel fills and assumes the shape of its container such that the material flows in both the main lumen L as well as in the lumens of the branching vessels MV1 and MV2 to completely seal and form a secondary artificial occlusion AO distal to the natural occlusion NO. Following the formation of the artificial occlusion AO, the guide wire 10 may be withdrawn (as shown in the remainder of the drawings) or the guide wire may be left in place to guide catheters or other apparatus to the treatment site.

With reference to FIG. 4, after formation of the artificial occlusion AO, an ablation tool 30 is shown ablating the natural occlusion NO. The ablation tool 30 is illustrated schematically as a rotary ablation tip such as that shown in U.S. Pat. No. 4,646,736. However, any ablation technique could be used (e.g., balloon angioplasty, stenting, jet or aspiration ablation, drug or chemical ablation or energy ablation such as ultrasound).

As a consequence of the ablation of the natural occlusion NO, a plurality of emboli E are formed. In the absence of the artificial occlusion AO, the emboli E could flow distally into the branching vessels MV1 and MV2 and lodge in smaller vessels in such a manner as to continue the ischemic condition of the tissue. If such occurred, the occlusion would now be in a plurality of much smaller vessels such that an ablation therapy may not be possible.

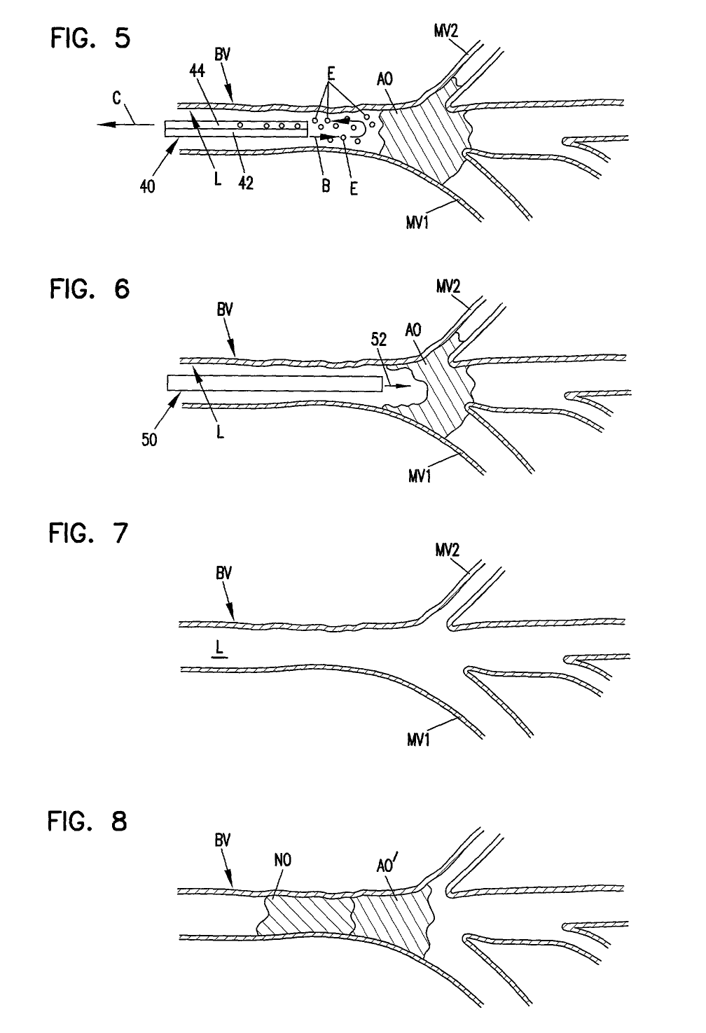

The artificial occlusion AO prevents the emboli E from flowing distally and retains the emboli E on the proximal side of the artificial occlusion AO. With the emboli E so restricted from distal flow, an emboli removal device 40 can be placed in the vessel BV as illustrated in FIG. 5. For ease of illustration, the emboli removal device 40 is shown as a double lumen catheter with a first lumen 42 for ejecting a jet of fluid and with a second lumen 44 connected to a suction. As a result, the ejected fluid flows in the direction of arrow B and is returned into the lumen 44 for flowing out of the lumen 44 in the direction of arrow C. In the process shown in FIG. 5, emboli E are entrained within the flowing fluid such that the emboli are captured and passed into the ejection lumen 44 for removal from the blood vessel BV. The emboli removal device could be any technique for recovering emboli. Such include aspiration or suction (e.g., U.S. Pat. Nos. 4,857,045; 6,022,336 and 5,938,645), any device to mechanically capture the emboli E or a drug maintained in the presence of the emboli for a sufficient residence time to dissolve the emboli.

Once the emboli E are removed, the treatment may be terminated and the hydrogel artificial occlusion AO may be permitted to simply dissolve. As the hydrogel dissolves, it dissolves completely so that it does not form emboli. As a consequence the present invention treats an unmanageable obstruction (i.e., the natural occlusion NO) by creating a manageable obstruction (the artificial occlusion AO). The artificial occlusion prevents undesirable emboli flow while the original natural occlusion is being removed. After removal of all the emboli E from the natural occlusion NO, the artificial occlusion AO may simply dissolve away resulting in complete patency of the lumen L and the lumen of the branching vessels MV1 and MV2.

While dissolution of the hydrogel artificial occlusion AO may be accomplished naturally by reason of the dissolution of the hydrogel in blood, the dissolution may be hastened to make the completion of the treatment more rapid. For example, with reference to FIG. 6, an ablation member 50 is shown within the lumen L for directing an ablation medium 52 at the artificial occlusion AO. The ablation member 50 could be any catheter and the ablation medium 52 could be any substance (including energy application) which results in a more rapid dissolution of the artificial AO.

For example, the ablation medium 52 may be a chemical solvent for chemically ablating the artificial occlusion AO. Alternatively, the catheter 50 may have an ultrasound transducer at its tip or a radio frequency emitter at its tip for emitting an energy selected to dissolve the hydrogel artificial occlusion AO. The hydrogel may also be formed to contain a solvent released by selection of a physician. For example, solvents can be contained in microbeads carried in the hydrogel. The microbeads can be ruptured by ultrasound application to release the solvent.

Once the artificial occlusion AO has dissolved through either dissolution in the body fluids without additional assistance or with assistance, e.g., through an ablation member 50, the lumen is now completely patent as illustrated in FIG. 7. Since emboli E have already been removed and since the artificial occlusion dissolves without long-term emboli, there are no further occlusions distal to the site of the original natural occlusion NO.

FIG. 8 illustrates an alternative embodiment where the artificial occlusion AO′ is positioned abutting a distal side of the natural occlusion NO. As a result, when the natural occlusion NO is being ablated, the physician will be able to determine that the natural occlusion NO has been fully ablated when the physician notes that ablated material of the artificial occlusion AO is being ejected also from the blood vessel BV. This could be accomplished by providing the artificial occlusion AO with a tracing member or material to act as a signature for the artificial occlusion material.

The artificial occlusion material may be provided with a radiopaque substance to identify its location. The formation of radiopaque hydrogels is discussed in Jayakrishnan et al., “Preparation and evaluation of radiopaque hydrogel microspheres based on PHEMA/iothalamic acid and PHEMA/iopanic acid as particulate emboli”, J. Biomedical Materials Research, pp. 993-1004 (August 1990). This article also discussed the use of hydrogel microspheres as particulate emboli in endovascular embolization. Certain hydrogels or polymers have been used to occlude blood vessels to treat tumors (U.S. Pat. No. 6,214,315), occlude a reproduction duct (U.S. Pat. No. 4,509,504) or plug diseased vessels (U.S. Pat. No. 5,258,042) or use of a porous hydrogel as an emboli filter (PCT International Publication WO 143662).

As an additional modification, the artificial occlusion may be laden with therapeutic agents such as drugs for treatment of distal tissue as the hydrogel dissolves. For example, the artificial occlusion material could be drug loaded with anti-coagulants, anti-thrombotic agents, anti-platelet agents, reperfusion injury prevention drugs, angiogenisus drugs or anti-spasmodic drugs such as calcium blocks. The use of hydrogels as drug carriers is discussed in Slepian et al., “Polymeric endoluminal gel paving: therapeutic hydrogel barriers and sustained drug delivery depots for local arterial wall biomanipulation”, Seminars in Interventional Cardiology, pp. 103-116 (March 1996). This article also describes use of hydrogel coatings on a wall of a vessel. Such coatings can be applied to the thrombus area after removal of the emboli. See, also, U.S. Pat. Nos. 5,714,159; 5,612,052 and 6,352,710.

To assist in desired placement of the artificial occlusion immediately distal to the natural occlusion NO, the distal tip 12 of the guide wire 10 can be provided with a sensing mechanism to indicate when the distal tip 12 has been passed through the natural occlusion NO. A non-limiting embodiment of such a sensing mechanism is illustrated in FIG. 9 as a strain gauge 12 positioned near port 14 and having electrical leads 22 extending proximally to equipment at the proximal end (not shown) of the guide wire 10. The strain gauge 20 can read a high strain as the guide wire 10 is being passed through the natural occlusion NO with the strain being relieved when the strain gauge 20 passes completely through the natural occlusion NO into the lumen L distal to the natural occlusion NO. Other sensing mechanisms are possible such as electrodes positioned near port 14 to measure an electrical conductivity or other change in parameters (such as pH) which would distinguish between the presence of the sensor within the natural occlusion NO and the presence of the sensor within the blood vessel lumen L distal to the natural occlusion NO.

In the embodiments described above, the invention has been illustrated as forming the artificial occlusion AO within the interior of the blood vessel. Additionally, the artificial occlusion AO can be formed by applying an occluding member to the exterior of the blood vessel BV distal to the natural occlusion NO. This is illustrated in FIGS. 10 and 11.

In FIGS. 10 and 11, a blood vessel such as a coronary artery CA is positioned in side-by-side, parallel alignment with a coronary vein CV. The artificial occlusion AO″ is formed by passing a balloon-tipped catheter 101 through the coronary vein CV and expanding the balloon 100 at a location distal to the natural occlusion such that the balloon and expanding coronary vein CV impinge upon and urge the coronary artery CA to close distal to the natural occlusion NO. This closure acts as the artificial occlusion AO″. The balloon can be held in place while the natural occlusion NO and resulting emboli are being removed. After such procedure, the balloon can be deflated and removed to restore the patency of the coronary artery CA.

Having disclosed the present invention a preferred embodiment, modifications and equivalents of the disclosed concepts should readily occur to one of ordinary skill in the art. It is intended that such modifications and equivalents shall be within the scope of the claim appended hereto.

What is claimed is:

1. A method of treating a body lumen having a natural occlusion at least partially occluding flow of body fluid from flowing proximally from said natural occlusion to locations distal to said natural occlusion, comprising:
obstructing said lumen with a conformal artificial occlusion distal to said natural occlusion;
ablating said natural occlusion to create a plurality of emboli of said natural occlusion on a proximal side of said artificial occlusion;
removing said emboli from said lumen; and
removing said artificial occlusion.

2. A method according to claim 1 wherein said obstructing is achieved by forming said artificial occlusion with said lumen.

3. A method according to claim 3 wherein said artificial occlusion is removed without substantial creation of permanent emboli.

4. A method according to claim 3 wherein said removing of said artificial occlusion is achieved by dissolving said artificial occlusion within said body fluid.

5. A method according to claim 3 wherein said removing of said artificial occlusion is achieved by dissolving said artificial occlusion by application of energy to said artificial occlusion.

6. A method according to claim 3 wherein said removing of said artificial occlusion is achieved by dissolving said artificial occlusion by application of chemical solvents to said artificial occlusion.

7. A method according to claim 6 wherein said solvents are contained within said artificial occlusion and selectively activated therein.

8. A method according to claim 3 wherein said removing of said artificial occlusion is achieved by mechanically ablating said artificial occlusion with said artificial occlusion formed from a material selected to form emboli dissolvable in said body fluid.

9. A method according to claims 3 wherein said artificial occlusion is formed from a hydrogel.

10. A method according to claim 9 wherein said hydrogel is delivered to said lumen distal to said natural occlusion in an unswelled state and swells to seal said lumen distal to said natural occlusion.

11. A method according to claim 2 wherein said artificial occlusion is laden with a therapeutic agent, said method further comprising releasing said agent into said lumen.

12. A method according to claim 2 wherein said artificial occlusion is radiopaque.

13. A method according to claim 11 wherein said agent is released during said removing of said artificial occlusion.

14. A method according to claim 1 wherein said lumen is a lumen of a blood vessel.

15. A method according to claim 14 wherein said blood vessel is a cerebral artery.

16. A method according to claim 14 wherein said blood vessel is a coronary artery.

17. An apparatus for treating a body lumen having a natural occlusion at least partially occluding flow of body fluid from flowing proximally from said natural occlusion to locations distal to said natural occlusion, comprising:
a delivery member sized to be passed through said body lumen proximal to said natural occlusion and having a distal end adapted to be passed through said natural occlusion to a position distal to said natural occlusion; and
said delivery member including a delivery port adjacent said distal end for delivery of an artificial occlusion into said lumen distal to said natural occlusion.

18. An apparatus according to claim 17 wherein said delivery member includes an internal cavity in communication with said delivery port and containing a material selected to form said artificial occlusion upon ejection of said material through said delivery port.

19. An apparatus according to claim 18 wherein said cavity is sized to contain a complete bolus of said material adjacent said delivery port.

20. An apparatus according to claim 18 wherein said cavity is a lumen through said delivery member connected to a source of said material at a proximal end of said delivery member.

21. An apparatus according to claim 18 further comprising an actuator for delivering said material from said delivery port.

22. An apparatus according to claim 18 wherein said material is susceptible to swelling within said lumen and said material is contained within said cavity in an unswelled state.

23. An apparatus according to claim 18 wherein said material is a hydrogel.

24. An apparatus according to claim 17 wherein said distal end includes a sensor for sensing when said distal end has passed through a distal side of said natural occlusion.

25. An apparatus according to claim 24 wherein said sensor includes a member for sensing a resistance of said natural occlusion to movement of said distal end through said natural occlusion.

26. An apparatus according to claim 24 wherein said sensor includes a member for sensing a change in a characteristic parameter between said natural occlusion and said body fluid.

27. An apparatus according to claim 18 wherein said material is dissolvable within said body fluid.

28. An apparatus according to claim 18 wherein said material is dissolvable by application of energy to said artificial occlusion.

29. An apparatus according to claim 18 wherein said material is dissolvable by application of chemical solvents to said artificial occlusion.

30. An apparatus according to claim 30 wherein said solvents are contained within said material and selectively activated therein.

31. An apparatus according to claim 18 wherein said material is removably by mechanically ablating said artificial occlusion with said material selected to form emboli dissolvable in said body fluid.

32. An apparatus according to claim 18 wherein said material is laden with a therapeutic agent.

33. An apparatus according to claim 18 wherein said material is radiopaque.

34. An apparatus according to claim 17 wherein said lumen is a lumen of a blood vessel.

35. An apparatus according to claim 34 wherein said blood vessel is a cerebral artery.

36. An apparatus according to claim 34 wherein said blood vessel is a coronary artery.

37. A kit for treating a body lumen having a natural occlusion at least partially occluding flow of body fluid from flowing proximally from said natural occlusion to locations distal to said natural occlusion, comprising:
an occlusion-creating member for obstructing said lumen with a material creating a conformal artificial occlusion distal to said natural occlusion;
an ablator for ablating said natural occlusion to create a plurality of emboli of said natural occlusion on a proximal side of said artificial occlusion; and
an emboli-removing member for removing said emboli from said lumen.

38. A kit according to claim 37 wherein said obstruction-creating member includes:
a delivery member sized to be passed through said body lumen proximal to said natural occlusion and having a distal end adapted to be passed through said natural occlusion to a position distal to said natural occlusion; and
said delivery member including a delivery port adjacent said distal end for delivery of a artificial occlusion into said lumen distal to said natural occlusion.

39. A kit according to claim 38 wherein said delivery member includes an internal cavity in communication with said delivery port and containing said material selected to form said artificial occlusion upon ejection of said material through said delivery port.

40. A kit according to claim 38 wherein said cavity is sized to contain a complete bolus of said material adjacent said delivery port.

41. A kit according to claim 38 wherein said cavity is a lumen through said delivery member connected to a source of said material at a proximal end of said delivery member.

42. A kit according to claim 38 further comprising an actuator for delivering said material from said delivery port.

43. A kit according to claim 38 wherein said material is susceptible to swelling within said lumen and said material is contained within said cavity in an unswelled state.

44. A kit according to claim 37 wherein said material is a hydrogel.

45. A kit according to claim 38 wherein said distal end includes a sensor for sensing when said distal end has passed through a distal side of said natural occlusion.

46. A kit according to claim 45 wherein said sensor includes a member for sensing a resistance of said natural occlusion to movement of said distal end through said natural occlusion.

47. A kit according to claim 45 wherein said sensor includes a member for sensing a change in a characteristic parameter between said natural occlusion and said body fluid.

48. A kit according to claim 37 wherein said material is dissolvable within said body fluid.

49. A kit according to claim 37 wherein said material is dissolvable by application of energy to said artificial occlusion.

50. A kit according to claim 37 wherein said material is dissolvable by application of chemical solvents to said artificial occlusion.

51. A kit according to claim 50 wherein said solvents are contained within said material and selectively activated therein.

52. A kit according to claim 37 wherein said material is removably by mechanically ablating said artificial occlusion with said material selected to form emboli dissolvable in said body fluid.

53. A kit according to claim 37 wherein said material is laden with a therapeutic agent.

54. A kit according to claim 37 wherein said material is radiopaque.

55. A kit according to claim 37 wherein said lumen is a lumen of a blood vessel.

56. A kit according to claim 55 wherein said blood vessel is a cerebral artery.

57. A kit according to claim 55 wherein said blood vessel is a coronary artery.

58. A method of treating a first blood vessel defining a first lumen having a natural occlusion at least partially occluding flow of blood from flowing proximally from said natural occlusion to locations distal to said natural occlusion and wherein a second blood vessel having a second lumen resides adjacent said first blood vessel distal to said natural occlusion, comprising:
advancing a first member through said second lumen to a position adjacent said first vessel distal to said natural occlusion;
urging said member against a wall of the second vessel to impinge upon the first vessel urging said first vessel to form an artificial occlusion distal to said natural occlusion;
ablating said natural occlusion to create a plurality of emboli of said natural occlusion on a proximal side of said artificial occlusion;
removing said emboli from said lumen; and
removing said artificial occlusion.
